(19) United States
(12) Patent Application Publication
(10) Pub. No.: US 2009/0264937 A1
(43) Pub. Date: Oct. 22, 2009

(54) BONE SCREW FOR PROVIDING DYNAMIC TENSION

(75) Inventor: Russell M. Parrott, Warsaw, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER, INC., Warsaw, IN (US)

(21) Appl. No.: 12/106,803

(22) Filed: Apr. 21, 2008

(51) Int. Cl. A61B 17/04

(52) U.S. Cl. 606/305; 606/301

(57) ABSTRACT

A bone screw including a head portion, an intermediate portion, and a threaded portion. The intermediate portion further includes a wave-type spring formed therein. In one exemplary embodiment, the wave-type spring is formed to have a lattice structure. In one exemplary embodiment, the wave-type spring of the intermediate portion is formed by laser cutting. By providing a wave-type spring in the intermediate portion of the bone screw of the present invention, the bone screw provides dynamic tension to maintain a bone plate or other orthopedic device in its desired position substantially adjacent a bone.

### BACKGROUND

1. Field of the Invention

The present invention relates to orthopedic devices and, particularly, to bone screws for use with orthopedic devices.

2. Description of the Related Art

Orthopedic devices, such as bone plates, may be used to maintain opposing portions of fractured bones substantially stationary relative to one another. For example, a bone plate may be formed as an elongate body having apertures extending therethrough and may be positioned to extend across the fracture line in a bone. Once positioned, bone screws may be inserted through the apertures to secure the bone plate to the fragments of the bone and maintain the opposing portions of the fractured bone in compression against one another.

Once inserted, the tension on the bone screw is initially large enough to maintain the opposing portions of the fractured bone and/or the bone plate in compression against one another. However, the opposing portions of the fracture bone may also undergo stress relaxation lessening the tension on the bone screw. Specifically, bone is a viscoelastic material and, as a result, it undergoes stress relaxation after a stress, e.g., the forces experienced during insertion of the screw, has been encountered by the bone. As a result of the stress relaxation of the bone, the tension on the screw decreases and, correspondingly, the force holding the bone fragments and/or bone plate together decreases.

In order to ensure that the bone fragments and/or bone plate maintain substantially consistent contact with one another after a bone undergoes stress relaxation, dynamic tension bone screws may be utilized. Dynamic tension bone screws currently include a coiled spring portion that allows the length of the bone screw to increase during insertion into a bone by stretching of the spring. Thus, as the bone undergoes stress relaxation, which results in a reduction in the tension exerted on the bone screw by the bone and the bone plate, the coiled spring portion of the bone screw will contract. This causes the bone screw to decrease in length and allows the bone plate to maintain consistent contact with the bone.

While dynamic tension bone screws according to known designs are effective, they utilize coiled springs to provide the dynamic tension. As a result, the springs of these bone screws substantially decrease the axial rigidity of the bone screws along the length of the springs. This allows for the bone screws to bend and to deviate from a straight line during insertion into a bone. More importantly, the amount of torque that can be applied to known dynamic tension bone screws is substantially limited. Specifically, if these bone screws are over-torqued, the coils of the springs formed therein may be displaced from their desired position, e.g., may expand outwardly, and the bone screw may become unusable.

### SUMMARY OF THE INVENTION

The present invention relates to orthopedic devices and, particularly, to bone screws for use with orthopedic devices. In one exemplary embodiment, the bone screw of the present invention includes a head portion, an intermediate portion, and a threaded portion. The intermediate portion further includes a wave-type spring formed therein. In one exemplary embodiment, the wave-type spring is formed to have a lattice structure. By providing a wave-type spring in the intermediate portion of the bone screw of the present invention, the bone screw provides dynamic tension to maintain a bone plate or other orthopedic device in its desired position substantially adjacent a bone and, when spanning a bone fracture, substantially continuously draws the bone fragments together.

In one exemplary embodiment, the wave-type spring of the intermediate portion of the bone screw is formed by a plurality of arms connected to one another at at least two discrete locations, which define junction points therebetween. As a result of the connection between the plurality of arms forming the wave-type spring, the bone screw of the present invention allows for a greater torque to be applied to the bone screw of the present invention without deforming the same.

In another exemplary embodiment, the bone screw of the present invention may include an internal bore and an alignment pin configured to travel within the internal bore. Thus, as the length of the bone screw is increased or decreased, the alignment pin travels along the internal bore and may contact the wall defining the internal bore. This contact between the alignment pin and the wall defining the internal bore provides additional axial rigidity to the bone screw. In another exemplary embodiment, the bone screw of the present invention may include a sleeve extending around at least a portion of the intermediate portion of the bone screw. The use of a sleeve also provides additional axial rigidity to the bone screw.

In one form thereof, the present invention provides a bone screw including: a head; and a shaft, the shaft including: a threaded portion having a thread defining a major diameter; and an intermediate portion positioned between the threaded portion and the head portion, the intermediate portion comprising a wall formed between an exterior surface of the shaft and an interior surface of the shaft, the wall defining a hollow portion of the shaft and comprising a wave spring.

In another form thereof, the present invention provides a method of securing a bone to an adjacent member, the method including the steps of: providing a bone screw including: a head; and a shaft, the shaft including: a threaded portion having a thread defining a major diameter; and an intermediate portion positioned between the threaded portion and the head portion, the intermediate portion comprising a wall formed between an exterior surface of the shaft and an interior surface of the shaft, the wall defining a hollow portion of the shaft and comprising a wave spring; advancing the threaded portion of the bone screw into the bone; contacting the head of the bone screw with the adjacent member; and expanding the intermediate portion of the bone screw.

In yet another form thereof, the present invention provides a bone screw including: a head; and a shaft including compression and expansion means for allowing for expansion and contraction of the intermediate portion while contemporaneously resisting torsion during both clockwise and counter-clockwise rotation of the shaft.

### BRIEF DESCRIPTION OF THE DRAWINGS

The above-mentioned and other features and advantages of this invention, and the manner of attaining them, will become more apparent and the invention itself will be better understood by reference to the following descriptions of embodiments of the invention taken in conjunction with the accompanying drawings, wherein:

FIG. 1 is a cross-sectional view of a bone and a bone plate and a perspective view of bone screws according to the present invention, wherein the bone screws are depicted as securing the bone plate to the bone and opposing portions of bone to one another;

FIG. 2 is a perspective view of one of the bone screws of FIG. 1;

FIG. 3 is a fragmentary, perspective view of an intermediate portion of the bone screw of FIG. 2 in an expanded state;

FIG. 4 is a fragmentary, perspective view of an intermediate portion of the bone screw of FIG. 2 wherein the intermediate portion is in a substantially unexpanded state;

FIG. 5 is a perspective view of a bone screw according to another embodiment of the present invention;

FIG. 6 is a cross-sectional view of the bone screw of FIG. 5 taken along line 6-6 of FIG. 5;

FIG. 7 is a perspective view of a bone screw according to another exemplary embodiment including a sleeve positioned thereon and depicted in cross-section; and

FIG. 8 is a diagram of 180° out-of-phase sinusoidal waves.

Corresponding reference characters indicate corresponding parts throughout the several views. The exemplifications set out herein illustrate preferred embodiments of the invention and such exemplifications are not to be construed as limiting the scope of the invention in any manner.

### DETAILED DESCRIPTION

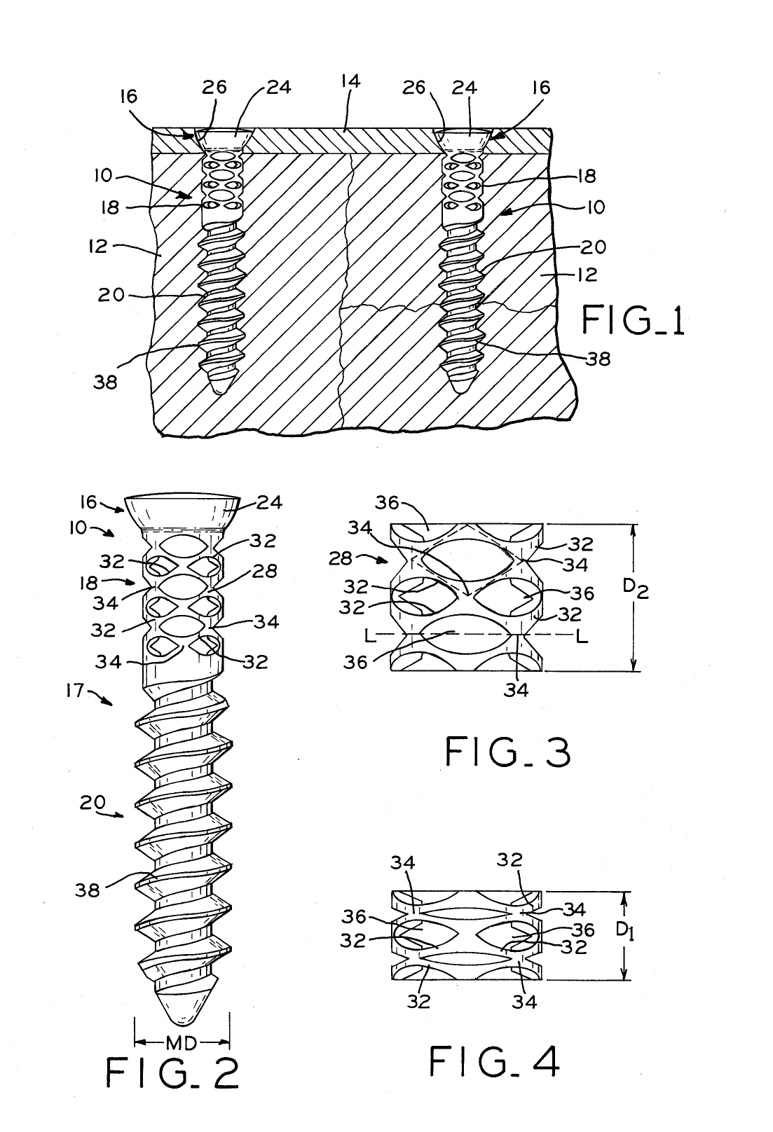

Referring to FIG. 1, bone screws 10 are shown secured within opposing portions of fractured bone 12. In this position, bone screws 10 function to retain bone plate 14 substantially adjacent bone 12, as described in detail below, and also function to retain opposing portions of fractured bone 12 adjacent one another. Referring to FIG. 2, bone screws 10 include head portion 16 and shaft 17, which includes intermediate portion 18 and threaded portion 20. Head portion 16 of bone screw 10 interacts with the surface of bone plate 14 to retain bone plate 14 against bone 12. In one exemplary embodiment, bone screw 10 is cannulated. In order to provide dynamic tension, intermediate portion 18 may be expanded during implantation of bone screws 10. Intermediate portion 18 is shown in an expanded state in FIG. 3, for example. Then, as bone 12 begins to undergo stress relaxation, the tension on bone screws 10 decreases. Specifically, bone, such as bone 12, is a viscoelastic material and, as a result, it undergoes stress relaxation after a stress, e.g., the forces experienced during insertion of bone screw 14, has been encountered by the bone. As a result of the stress relaxation experience by bone 12, the tension on bone screw 10 decreases and, correspondingly, the force holding the fragments of bone 12 and/or bone plate 14 together decreases. As the tension on bone screws 10 decreases, intermediate portion 18 may contract to a normal, unexpanded state of equilibrium, such as the state shown in FIG. 4. As a result of the contraction of intermediate portion 18, bone plate 14 is pulled substantially adjacent bone 12, as described in detail below.

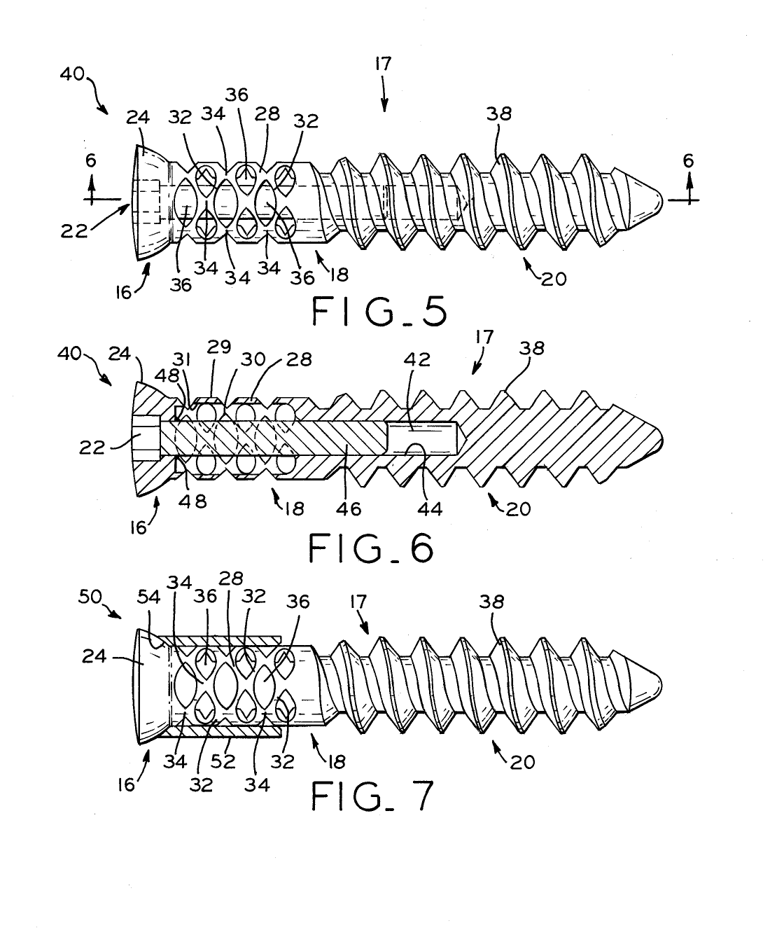

Turning to specific aspects of bone screw 10, bone screw 10 may be machined from any biocompatible metal, such as titanium, alloys of titanium, such as Ti-6Al-4V, cobalt chromium, or Nitinol. Additionally, head portion 16 of bone screw 10 may include a drive tool receiving aperture 22, shown in FIG. 5, configured to receive a corresponding drive tool. For example, drive tool receiving aperture 22 may be defined by a series of walls forming a substantially hexagonal shaped aperture configured to receive a corresponding substantially hexagonal shaped male portion of a drive tool. While described and depicted herein as being substantially hexagonal shaped, drive tool receiving aperture 22 may have any other shape configured for engagement with a corresponding drive tool. In one exemplary embodiment, exterior surface 24 of head portion 16 may define a substantially hexagonal shape. In this embodiment, a female drive tool having a substantially hexagonal shape corresponding to the substantially hexagonal shape of exterior surface 24 may be used to impart torque to bone screw 10 by mating therewith. Similar to drive tool receiving aperture 22, exterior surface 24 of head portion 16 may take any shape configured to mate with a corresponding drive tool for imparting torque to bone screw 10. Additionally, as shown in FIG. 2, exterior surface 24 of head portion 16 defines a substantially tapered surface configured to engage the surface of bone plate 14 defining apertures 26 extending therethrough.

Referring again to FIGS. 2, intermediate portion 18 of bone screw 10 includes wall 28 defined between exterior surface 29 (FIG. 6) of shaft 17 and interior surface 31 (FIG. 6) of shaft 17. The outside diameter of intermediate portion 18 could be larger than the maximum outside diameter of threaded portion 20, but is more preferably smaller. This outside diameter could range between 2 mm and 8 mm, more preferably between 4 mm and 6 mm. The inside diameter could be adjusted to the outside diameter to maintain the thickness of wall 28 to provide enough strength to resist the applied forces to the screw during insertion and throughout its implant life. This wall thickness should be a minimum of one millimeter. Wall 28 of intermediate portion 18 defines hollow portion 30 of bone screw 10, as best seen in FIG. 6, and includes a plurality of arms 32. Each of arms 32 are configured to contact at least one opposing arm 32 at junctions 34. By connecting arms 32 at junctions 34, wall 28 defines a lattice structure, i.e., wall 28 has a regular geometric arrangement of arms 32 and junctions 34 over the area defined by wall 28. Additionally, defined between arms 32 and junctions 34 are openings 36, which are formed in wall 28. The formation of openings 36 allows for the expansion and contraction of intermediate portion 18. In one exemplary embodiment, openings 36 are laser cut into wall 28. In another exemplary embodiment, openings 36 are milled into wall 28.

In one exemplary embodiment, arms 32 and junctions 34 of wall 28 cooperate to define a wave-type spring that is substantially similar to 180° out-of-phase sinusoidal waves, such as those shown in FIG. 8. For example, referring to FIGS. 3 and 8, baseline D of FIG. 8 is substantially similar to line L-L of FIG. 3, which is drawn perpendicular to the longitudinal axis of bone screw 10 and through at least two junctions 34. Thus, the spaces bounded by the opposing sinusoidal waves in FIG. 8 are substantially similar to openings 36 of wall 28 in FIG. 3. Additionally, each intersection of opposing sinusoidal waves at baseline D of FIG. 8 is substantially similar to each of junctions 34 on line L of FIG. 3. To increase the length of wall 28, additional pairs of 180° out-of-phase sinusoidal waves may be aligned one atop another. In one exemplary embodiment, the lattice structure defined by wall 28 has a substantially diamond-shaped geometrical configuration. For example, as shown in dashed lines in FIG. 3, a diamond shape may be formed by arms 32 by designing arms 32 to have substantially straight sides, instead of substantially curved sides. As a result, openings 36 would be defined by substantially straight boundary walls instead of substantially curved boundary walls to define a diamond-shaped geometrical configuration.

Referring to FIG. 2, threaded portion 20 is positioned at the distal end of shaft 17 of bone screw 10 and includes thread 38 positioned thereon. Thread 38 has a major diameter MD measured from the outermost portion of opposing sides of thread 38. In one exemplary embodiment, thread 38 has a major diameter of substantially between 2.0 to 8.0 mm. Thread 38 may be a viable pitch thread or may have a design substantially similar to the thread design utilized with cancellous bone screws and/or cortical bone screws, for example. Additionally, in one exemplary embodiment, thread 38 may be self-tapping, eliminating the need to tap a hole formed in the bone prior to insertion of bone screw 10 into the hole. In another exemplary embodiment, thread 38 may be self-drilling, eliminating the need to drill a pilot hole to facilitate the insertion of bone screw 10 into a bone.

Referring to FIG. 1 and as discussed briefly above, bone screw 10 may be used to secure an orthopedic device, such as bone plate 14, to a bone, such as bone 12. Specifically, bone plate 14 is positioned substantially adjacent opposing portions of fractured bone 12 to secure the opposing portions of fractured bone 12 adjacent to one another. Once bone plate 14 is positioned substantially adjacent bone 12, bone screws 10 may be inserted through apertures 26 in bone plate 14. In one exemplary embodiment, pilot holes may be drilled prior to inserting bone screws 10 to facilitate the insertion of bone screws 10. Additionally, in order to insert bone screws 10, a drive tool have a male portion corresponding to drive tool receiving aperture 22 (FIG. 5) may be received therein. Torque is then imparted to the drive tool, which transfers the torque to bone screw 10 to cause corresponding rotation of bone screw 10. As bone screw 10 rotates, threads 38 engage bone 12 and the interaction between thread 38 and bone 12 advances bone screw 10 into the bone.

As torque continues to be applied to the drive tool and bone screw 10 continues to advance into bone 12, exterior surface 24 of head portion 16 of bone screw 10 contacts the surface defining aperture 26 in bone plate 14. As a result of the interaction between the surface defining aperture 26 in bone plate 14 and exterior surface 24 of head portion 16, additional advancement of head portion 14 along the longitudinal axis of bone screw 10 is prevented. Thus, as torque is continued to be imparted to bone screw 10 and bone screw 10 correspondingly rotated, thread 38 and, correspondingly, threaded portion 20 and intermediate portion 18 of shaft 17 of bone screw 10 will continue to advance into bone 12. As intermediate portion 18 and threaded portion 20 continue to advance, intermediate portion 18 will begin to stretch to the expanded position shown in FIG. 3. In one exemplary embodiment, intermediate portion 18 may stretch from its equilibrium state shown in FIG. 4 and having a distance D₁ to an expanded state shown in FIG. 3 and having a distance D₂. In one exemplary embodiment, D₂ is between about 0 and 2 mm greater than D₁ and results in a corresponding increase in the overall length of bone screw 10.

In this position, bone screw 10 exerts a restoring, contractive force on bone plate 14 and bone 12 as a result of the stretching of intermediate portion 18, drawing bone plate 14 and bone 12 substantially adjacent one another. Thus, once positioned as shown in FIG. 1, bone screw 10 will act to keep bone plate 14 in a position substantially adjacent to bone 12. Specifically, if bone 12 undergoes stress relaxation, the tension on bone screw 10 may decrease and, correspondingly, the force holding the fragments of bone 12 and/or bone plate 14 together decreases. When this occurs, the restorative, contractive force resulting from the stretching of the wave-type spring of intermediate portion 18, described in detail above, will draw head portion 16, and correspondingly bone plate 14, and threaded portion 18, and correspondingly bone 12, toward one another. Bone screw 10 will continue to apply a restorative, contractive force on bone plate 14 and bone 12 until intermediate portion 18 returns to its normal, unexpanded state of equilibrium, shown in FIG. 4.

Advantageously, by utilizing the wave-type spring design of intermediate portion 18, higher amounts of torque may be applied to bone screw 10 than a traditional dynamic tension screw. Specifically, in known dynamic tension screws, the restorative, contractive force is provided by a coiled spring. Thus, when torque is exerted on the shaft of the screw, the coils of the spring may begin to uncoil and/or unscrew, which may cause deformation of the bone screw and render it unsuitable for its intended purpose. Additionally, the bone screw design of the present invention substantially lessens the difficulty encountered in removing the bone screw from a bone, as the wave-type spring design of the present bone screw resists torsion in both a clockwise and counter-clockwise direction. Specifically, in contrast to known dynamic tension bone screws, junctions 34 prevent the wave-type spring of bone screw 10 from uncoiling during either implantation or removal. Thus, bone screw 10 allows for the use of higher torque during implantation and also eases removal of bone screw 10 from a bone.

Referring to FIGS. 5 and 6, another exemplary embodiment of bone screw 10 is shown as bone screw 40. Bone screw 40 has several parts that are identical or substantially identical to corresponding parts of bone screw 10 of FIGS. 1-4 and identical reference numerals have been used to identify identical or substantially identical parts therebetween. Referring to FIG. 6, threaded portion 20 of bone screw 40 includes internal bore 42 defined by interior wall 44. Alignment pin 46 is positioned within internal bore 42. In order to position alignment pin 46 within internal bore 42, an opening is formed in head portion 16 of bone screw 40 and alignment pin 46 is inserted therethrough. Alignment pin 46 may then be welded at welds 48 to secure alignment pin 46 to head portion 16. In another exemplary embodiment, alignment pin 46 may be secured to head portion 16 by a snap-fit. Additionally, alignment pin 46 may be connected to head portion 16 using other known fasteners or techniques, such as brazing, for example.

As shown in FIGS. 5 and 6, bone screw 40 is in a substantially expanded, stretched state. However, in its normal, unexpanded state of equilibrium, alignment pin 46 may be seated at the bottom of internal bore 42. During expansion and contraction of intermediate portion 18 of bone screw 40, alignment pin 46 travels along internal bore 42. As alignment pin 46 travels along internal bore 42, it may engage interior wall 44. The interaction between alignment pin 46 and interior wall 44 provides additional axial rigidity to bone screw 40. Specifically, in the event that a force was placed on bone screw 40 that would cause bone screw 40 to bend in a manner that would make its longitudinal axis deviate from a substantially straight line, alignment pin 46 would contact interior wall 44 of interior bore 42 to resist this movement. This additional axial rigidity helps bone screw 40 remain substantially straight during insertion and removal, for example.

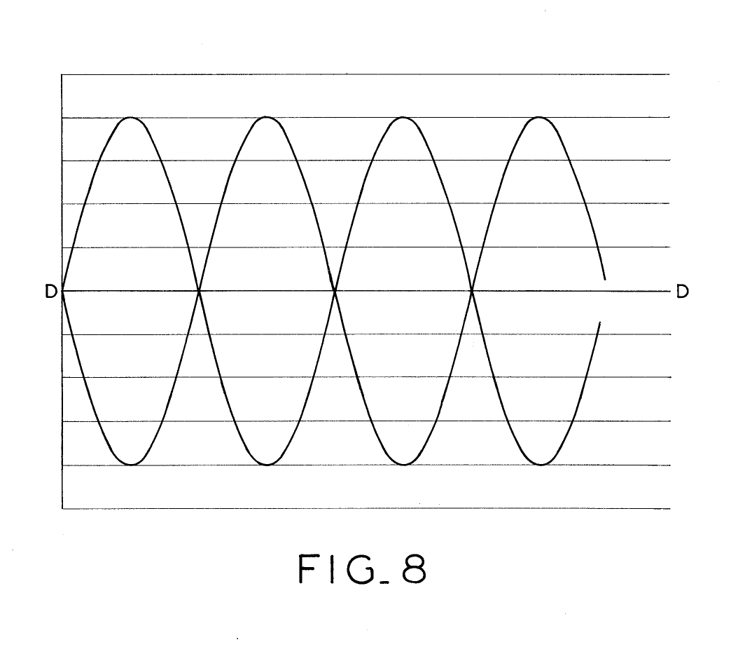

Referring to FIG. 7, another exemplary bone screw is shown as bone screw 50. Bone screw 50 has several parts that are identical or substantially identical to corresponding parts of bone screw 10 of FIGS. 1-4 and identical reference numerals have been used to identify identical or substantially identical parts therebetween. As shown in FIG. 7, bone screw 50 includes an external reinforcement member, such as sleeve 52 positioned thereon. Specifically, sleeve 52 is inserted over threaded portion 20 of bone screw 50 and advanced to contact exterior surface 24 of head portion 16. Once in this position, sleeve 52 is secured to head portion 16. For example, sleeve 52 may be secured to head portion 15 by welding or brazing, for example. In another exemplary embodiment, sleeve 52 is positioned over threaded portion 20 of bone screw 50 but is not secured to head portion 16. In one exemplary embodiment sleeve 52 includes tapered surface 54 having a taper that is substantially similar to the taper of exterior surface 24 of head portion 16. Thus, tapered surface 54 of sleeve 52 flushingly engages exterior surface 24 of head portion 16. Additionally, in one exemplary embodiment, sleeve 52 has a length that allows sleeve 52 to extend from head portion 16 to substantially entirely cover intermediate portion 18 when bone screw 50 is in an expanded state. Alternatively, in other exemplary embodiment, sleeve 52 has a length that is insufficient to substantially entirely cover intermediate portion 18 when bone screw 50 is in an expanded state.

Similar to alignment pin, by providing sleeve 52 on bone screw 50, the axial rigidity of bone screw 50 is increased. Specifically, in the event that a force was placed on bone screw 50 that would cause bone screw 50 to bend in a manner that would make its longitudinal axis deviate from a substantially straight line, sleeve 52 would contact exterior surface 29 of wall 28 of intermediate portion 18 to resist this movement. This additional axial rigidity helps bone screw 50 remain substantially straight during insertion and removal, for example.

Alternatively, in another exemplary embodiment, an external reinforcement member in the form of a plurality of elongate bars (not shown) is used. The plurality of elongate bars may be secured to head portion 16 of bone screw 50. The bars may extend down over at least of portion of intermediate portion 18 of bone screw 50 in a substantially similar manner as sleeve 52. In one exemplary embodiment, three bars use positioned about intermediate portion 18 and are space from one another by approximately 120 degrees. By properly arranging the plurality of elongate bars, an improvement in the axial rigidity of bone screw 50 that is substantially similar to the improvement achieved by sleeve 52 may be obtained.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A bone screw comprising:
a head; and
a shaft, said shaft comprising:
a threaded portion having a thread defining a major diameter; and
an intermediate portion positioned between said threaded portion and said head portion, said intermediate portion comprising a wall formed between an exterior surface of said shaft and an interior surface of said shaft, said wall defining a hollow portion of said shaft and comprising a wave spring.

2. The bone screw of claim 1, wherein said wave spring comprises a geometrical configuration substantially similar to 180 degree out of phase adjacent sinusoidal waves.

3. The bone screw of claim 1, wherein said wave spring further comprises a lattice structure.

4. The bone screw of claim 3, wherein said lattice structure comprises a plurality of interconnected arms, wherein said plurality of interconnected arms are joined with one another to define at least two discrete junction points therebetween.

5. The bone screw of claim 3, wherein said wave spring further comprises a substantially diamond-shaped geometrical configuration.

6. The bone screw of claim 1, wherein said head portion further comprises a drive tool receiving portion.

7. The bone screw of claim 1, wherein said threaded portion further comprises an internal wall defining an internal bore and said head portion further comprises an alignment pin, said alignment pin dimensioned for receipt within said internal bore of said threaded portion, said alignment pin having a length, wherein said length is sufficient to maintain at least a portion of said alignment pin within said internal bore of said threaded portion irrespective of whether said bone screw is in an expanded state and an equilibrium state.

8. The bone screw of claim 1, further comprising a external reinforcement member to provide axial rigidity to the bone screw.

9. The bone screw of claim 8, wherein said external reinforcement member comprises a sleeve secured to said head portion of said bone screw, said sleeve having a length, said length sufficient to extend from said head portion of said bone screw substantially entirely over said intermediate portion of said bone screw when said bone screw is in an equilibrium state.

10. The bone screw of claim 1, wherein said wave-type spring of said intermediate portion further comprises a laser cut plurality of interconnected arms.

11. A method of securing a bone to an adjacent member, the method comprising the steps of:
providing a bone screw comprising:
a head; and
a shaft, the shaft comprising:
a threaded portion having a thread defining a major diameter; and
an intermediate portion positioned between the threaded portion and the head portion, the intermediate portion comprising a wall formed between an exterior surface of the shaft and an interior surface of the shaft, the wall defining a hollow portion of the shaft and comprising a wave spring;
advancing the threaded portion of the bone screw into the bone;
contacting the head of the bone screw with the adjacent member; and
expanding the intermediate portion of the bone screw.

12. The method of claim 11, wherein the adjacent member comprises one of a bone plate, a second bone, and an orthopedic implant.

13. The method of claim 11, wherein the wave spring further comprises a lattice structure.

14. The method of claim 13, wherein the lattice structure further comprises a plurality of interconnected arms, wherein said plurality of interconnected arms are joined with one another to define at least two discrete junction points therebetween.

15. The method of claim 13, wherein the lattice structure further comprises a geometrical configuration substantially similar to 180 degree out of phase adjacent sinusoidal waves.

16. The method of claim 13, wherein the lattice structure further comprises a substantially diamond-shaped geometrical configuration.

17. A bone screw comprising:
a head; and
a shaft including compression and expansion means for allowing expansion and contraction of said intermediate portion while contemporaneously resisting torsion during both clockwise and counter-clockwise rotation of said shaft.

18. The bone screw of claim 17, wherein said compression and expansion means comprise a wave spring.

19. The bone screw of claim 18, wherein said wave spring comprises a geometrical configuration substantially similar to 180 degree out of phase adjacent sinusoidal waves.

20. The bone screw of claim 1, wherein said wave spring further comprises a lattice structure, said lattice structure including a plurality of interconnected arms, wherein said plurality of interconnected arms are joined with one another to define at least two discrete junction points therebetween.
